(19) United States
(12) Patent Application Publication
(10) Pub. No.: US 2026/0191400 A1
(43) Pub. Date: Jul. 9, 2026

(54) TIP HOOD, ENDOSCOPE DEVICE, AND ENDOSCOPE SYSTEM

(71) Applicant: OLYMPUS MEDICAL SYSTEMS CORP., Tokyo (JP)

(72) Inventors: Yuma KASUYA, Tokyo (JP); Keita KIMURA, Tokyo (JP); Shinya TORII, Tokyo (JP); Masanori MAKI, Tokyo (JP)

(73) Assignee: OLYMPUS MEDICAL SYSTEMS CORP., Tokyo (JP)

(21) Appl. No.: 19/560,660

(22) Filed: Mar. 9, 2026

Related U.S. Application Data
(63) Continuation of application No. PCT/JP2023/033034, filed on Sep. 11, 2023.

(51) Int. Cl. A61B 1/00; A61B 1/015; A61B 18/14

(52) U.S. Cl. CPC A61B 1/00089; A61B 1/00006; A61B 1/00091; A61B 1/00097; A61B 1/015; A61B 18/14; A61B 2018/1412; A61B 2218/008

(57) ABSTRACT

A tip hood configured to be attached to a distal end portion of an endoscope. The tip hood includes a gas feed conduit configured to allow a predetermined gas to flow therethrough, a first gas feed outlet configured to feed the gas, supplied through the gas feed conduit, in a field-of-view direction of the endoscope, and a second gas feed outlet configured to feed the gas, supplied through the gas feed conduit, toward a distal end surface of the endoscope.

### CROSS REFERENCE TO RELATED APPLICATIONS

This application is a continuation of International Application No. PCT/JP2023/033034, filed on Sep. 11, 2023, the entire contents of which are incorporated herein by reference.

### BACKGROUND

### Field

The present disclosure relates to a tip hood, an endoscope device, and an endoscope system, which remove smoke generated when a treatment operation is performed.

### Related Art

An endoscope system, which includes an endoscope that captures an image of an object in a subject, a video processor that applies image processing to an observation image of the object picked up by the endoscope and outputs the processed observation image, and the like, has been widely used in medical fields, and the like.

In general, an endoscope is used by inserting an insertion portion into a subject, and configured to acquire an image with an image pickup apparatus disposed in a distal end portion of the insertion portion. In addition, an endoscope is used for performing a treatment operation for peeling and removing a tissue to be treated from a living tissue. The treatment operation is performed by causing a treatment instrument such as a high-frequency probe, which is inserted into an instrument channel through a treatment instrument insertion port provided at an operation portion, to protrude beyond a distal end portion.

In such a treatment operation, a field of view is sometimes obstructed by smoke generated when an affected area is cauterized with a high-frequency probe or the like. To address such circumstances, a configuration is known in which gas is fed from the distal end portion toward a smoke generation point.

For example, Japanese Patent Application Laid-Open Publication No. 2013-169380 discloses an endoscope device including a plurality of gas-feeding ports on a distal end surface of an insertion portion, and configured to inhibit dirt from adhering to an image pickup lens by feeding a gas from the plurality of gas-feeding ports.

### SUMMARY

According to aspects of the present disclosure, a tip hood is provided, which is configured to be attached to a distal end portion of an endoscope. The tip hood includes a gas feed conduit, a first gas feed outlet, and a second gas feed outlet. The gas feed conduit is configured to allow a predetermined gas to flow therethrough. The first gas feed outlet is configured to feed the gas, supplied through the gas feed conduit, in a field-of-view direction of the endoscope. The second gas feed outlet is configured to feed the gas, supplied through the gas feed conduit, toward a distal end surface of the endoscope.

According to aspects of the present disclosure, further provided is an endoscope device that includes an endoscope, a tip hood, a gas feed conduit, a first gas feed outlet, and a second gas feed outlet. The tip hood is configured to be attached to a distal end portion of the endoscope. The gas feed conduit is configured to allow a predetermined gas to flow therethrough. The first gas feed outlet is disposed at the tip hood and configured to feed the gas, supplied through the gas feed conduit, in a field-of-view direction of the endoscope. The second gas feed outlet is disposed at the tip hood and configured to feed the gas, supplied through the gas feed conduit, toward a distal end surface of the endoscope.

According to aspects of the present disclosure, further provided is an endoscope system that includes an endoscope, a tip hood, a gas feed conduit, a control device, a first gas feed outlet, and a second gas feed outlet. The tip hood is configured to be attached to a distal end portion of the endoscope. The gas feed conduit is configured to allow a predetermined gas to flow therethrough. The control device is configured to control supply of the gas to the gas feed conduit. The first gas feed outlet is disposed at the tip hood and configured to feed the gas, supplied through the gas feed conduit, in a field-of-view direction of the endoscope. The second gas feed outlet is disposed at the tip hood and configured to feed the gas, supplied through the gas feed conduit, toward a distal end surface of the endoscope.

### BRIEF DESCRIPTION OF DRAWINGS

FIG. 1 is a schematic configuration view showing a configuration of an endoscope system according to a first embodiment.

FIG. 2 is a perspective view showing a state where a tip hood is attached to a distal end portion.

FIG. 3 is a cross-sectional view showing the state where the tip hood is attached to the distal end portion.

FIG. 4 describes an arrangement relationship between a first gas feed hole and a second gas feed hole.

FIG. 5 is a front view in a case where the tip hood is attached to the distal end portion.

FIG. 6 shows a configuration of a tip hood according to a modified example of the first embodiment.

FIG. 7 shows a configuration of a smoke removal device in a second embodiment.

FIG. 8 shows a configuration of a tip hood in the second embodiment.

FIG. 9 shows a configuration in which gas discharge holes (gas exhaust ports) are provided in a gas discharge conduit (a gas exhaust conduit).

FIG. 10 shows a configuration of a tip hood in a third embodiment.

FIG. 11 shows a configuration of a smoke removal device in the third embodiment.

FIG. 12 shows configurations of a tip hood and a smoke removal device in a modified example of the third embodiment.

FIG. 13 shows a connection relationship between a smoke removal device and a high-frequency incision device in a fourth embodiment.

FIG. 14 shows a configuration in a case where start and stop of smoke removal is controlled by a foot switch.

FIG. 15 shows a configuration in a case where the start and stop of the smoke removal is controlled by a switch of an endoscope.

FIG. 16 shows a configuration of a tip hood in a fifth embodiment.

FIG. 17 shows a configuration of a tip hood in a sixth embodiment.

FIG. 18 shows a configuration of a smoke removal device in the sixth embodiment.

FIG. 19 shows a configuration of a tip hood in a seventh embodiment.

FIG. 20 shows a configuration of a smoke removal device in the seventh embodiment.

FIG. 21 shows a configuration of a smoke removal device in an eighth embodiment.

FIG. 22 shows a configuration of a tip hood in a modified example of the eighth embodiment.

### DESCRIPTION OF EMBODIMENTS

A configuration is known in which a cylindrical-shaped tip hood is attached to a distal end portion of an insertion portion. The tip hood is an element configured to be attached to a distal end of an endoscope, to keep an appropriate distance between an observation object and a lens surface of the endoscope. To meet such a characteristic, the tip hood has a shape protruding beyond a distal end surface of the endoscope.

Such a configuration has a problem that, when a gas is fed from the distal end portion of the insertion portion toward a smoke generation point, the pushed-out smoke flows to a distal end surface side of the endoscope, and the smoke is likely to stay inside the tip hood protruding beyond the distal end surface, which interferes with a field of view.

Hereinafter, embodiments of the present disclosure will be described with reference to drawings.

Note that the drawings based on each embodiment are schematic. The relationship between thicknesses and widths of respective parts, a ratio of thicknesses, and the like of the respective parts are different from the actual ones. The respective drawings include parts in which the relationships and ratios among the dimensions are different.

### First Embodiment

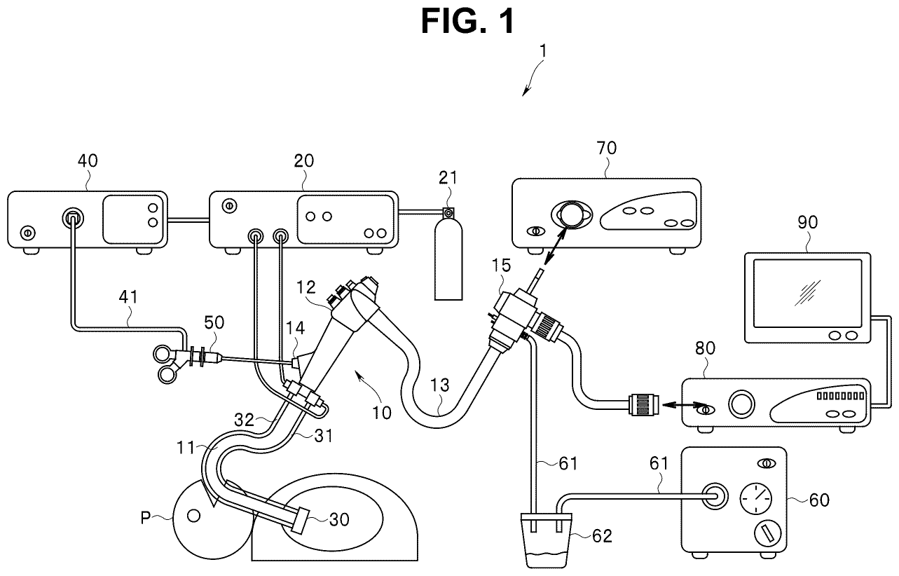

FIG. 1 is a schematic configuration view showing a configuration of an endoscope system according to a first embodiment. As shown in FIG. 1, an endoscope system 1 includes an endoscope 10, a smoke removal device 20, a tip hood 30, a high-frequency incision device 40, a treatment instrument 50, a suction device 60, a light source device 70, a video processor 80, and a monitor 90.

The endoscope 10 includes an insertion portion 11 configured to be inserted into a digestive tract of a patient P, an operation portion 12, and a universal cable 13.

The operation portion 12 includes a treatment instrument insertion port 14 through which the treatment instrument 50 such as a high-frequency probe is inserted. The universal cable 13 includes, at an end portion thereof, a connector portion 15. The endoscope 10 is connected to the light source device 70 and the video processor 80 through the connector portion 15.

In addition, the connector portion 15 is provided with a suction ferrule. The suction ferrule is connected to the suction device 60 through a suction tube 61. The suction tube 61 includes, at the middle portion thereof, a suction bottle 62. A liquid sucked by control of the suction device 60 is stored in the suction bottle 62.

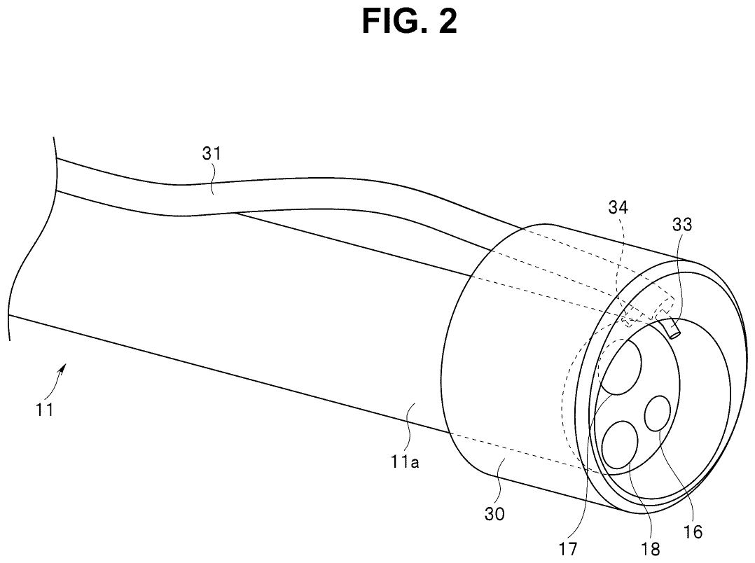

The tip hood 30 is attached to a distal end portion 11a of the insertion portion 11 (see FIG. 2). The tip hood 30 includes a gas feed conduit 31 and a gas discharge conduit (hereinafter also referred to as a gas exhaust conduit) 32. The gas feed conduit 31 and the gas exhaust conduit 32 are connected to the smoke removal device 20.

The smoke removal device 20 may include processing circuitry including one or more processors and one or more non-transitory computer-readable storage media. The smoke removal device 20 as a control device is configured to feed a predetermined gas into a digestive tract through the gas feed conduit 31, and discharge the predetermined gas through the gas exhaust conduit 32. A medical-use gas cylinder 21 is connected to the smoke removal device 20. Although the predetermined gas fed through the gas feed conduit 31 is not limited in particular, a CO₂ gas, which is supplied as a medical-use gas from the gas cylinder 21, is used in the present embodiment.

In general, an inside of a digestive tract is in a high humidity environment, and if high-temperature smoke flows into the tip hood 30, moisture condensation occurs on an objective lens 17 (see FIG. 2), which is likely to interfere with a field of view of the endoscope 10.

In the present embodiment, as will be described later, a gas sprayed from a second gas feed hole (hereinafter also referred to as a second gas feed outlet) 34 (see FIG. 2) may be a dried gas. With this, the inside of the tip hood 30 is always kept in a low humidity environment. Therefore, even if high-temperature smoke flows into the tip hood 30, moisture condensation can be inhibited from occurring on the objective lens 17.

In a surgery room, as a medical-use gas, a CO₂ gas is generally used. As the dried gas, the CO₂ gas as the medical-use gas is used. The CO₂ gas has a biological absorption rate higher than that of air, and even when a digestive tract is pressurized by gas feeding, a patient feels less pain. In addition, since the CO₂ gas is incombustible, even when gas is fed to a cauterization point from a first gas feed hole (hereinafter also referred to as a first gas feed outlet) 33 (see FIG. 2), there is no risk of fire and a procedure can be performed safely.

The high-frequency incision device 40 is connected to the treatment instrument 50, through a treatment instrument cable 41 for supplying high-frequency power. The treatment instrument 50 is inserted into a treatment instrument insertion channel in the insertion portion 11 from the treatment instrument insertion port 14 formed at the operation portion 12 of the endoscope 10. A user can perform treatment by causing the treatment instrument 50 to protrude beyond the distal end portion 11a of the insertion portion 11.

The light source device 70 supplies illumination light to the endoscope 10 through the universal cable 13. When the connector portion 15 is connected to the light source device 70, the illumination light is transmitted through a light guide fiber, not shown, to be emitted from an illumination lens provided at the distal end portion of the insertion portion 11.

The video processor 80 as an image processing apparatus converts an electric signal from an image pickup device provided at the distal end portion of the insertion portion 11 of the endoscope 10 into a video signal, and outputs the video signal to the monitor 90. On the screen of the monitor 90, an endoscopic image of an object captured by the endoscope 10 is displayed.

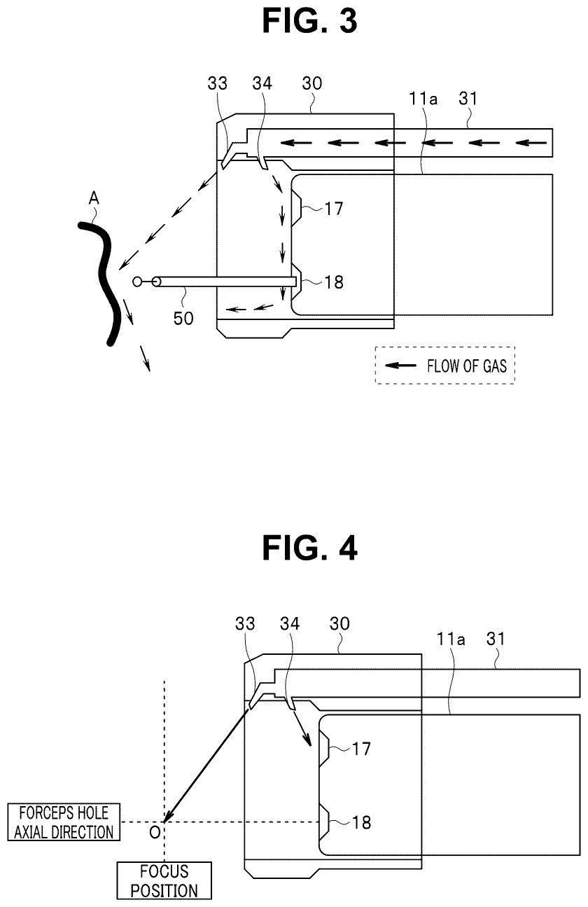

FIG. 2 is a perspective view showing a state where the tip hood is attached to the distal end portion. FIG. 3 is a cross-sectional view showing the state where the tip hood is attached to the distal end portion. Note that illustration of the gas exhaust conduit 32 is omitted in FIG. 2 and FIG. 3.

As shown in FIG. 2, the distal end portion of the insertion portion 11 includes an illumination lens 16, the objective lens 17, and a forceps hole (hereinafter also referred to as an instrument channel) 18.

Furthermore, the tip hood 30 includes, in addition to the gas feed conduit 31 for transmitting the above-described predetermined gas, the first gas feed hole 33 from which the gas is fed in a field-of-view direction of the endoscope 10 through the gas feed conduit 31, and the second gas feed hole 34 from which the gas is fed toward the distal end side (e.g., a distal end surface) of the endoscope 10 through the gas feed conduit 31. The first gas feed hole (the first gas feed outlet) 33 and the second gas feed hole (the second gas feed outlet) 34 are bent from an axial direction of the gas feed conduit 31 toward an inner surface. In other words, the first gas feed outlet 33 and the second gas feed outlet 34 are angled toward an interior of the tip hood 30 with respect to the axial direction of the gas feed conduit 31.

Smoke generated by a treatment operation by the treatment instrument 50 and staying in the vicinity of a diseased part A, i.e., a digestive tract wall is pushed out by the gas fed from the first gas feed hole 33. With this, the smoke that has flowed into the tip hood 30 is pushed out by the gas fed from the second gas feed hole 34. As a result, the smoke generated in the field-of-view range of the endoscope 10 can be efficiently removed.

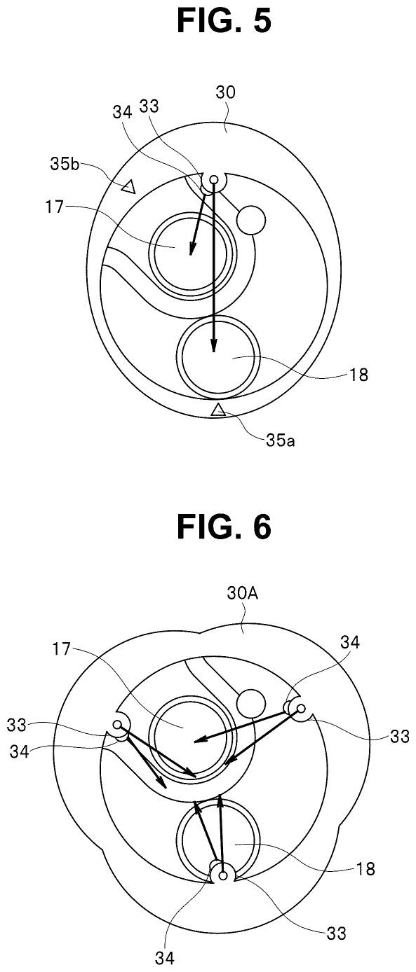

Note that, in order to remove the smoke efficiently, the first gas feed hole 33 and the second gas feed hole 34 may be arranged as shown in FIGS. 4 and 5.

FIG. 4 describes an arrangement relationship between the first gas feed hole and the second gas feed hole 34. FIG. 5 is a front view in the case where the tip hood is attached to the distal end portion.

As shown in FIG. 4, the first gas feed hole (the first gas feed outlet) 33 is arranged such that the gas is fed toward an intersection O of an axial direction (more specifically, a central axis) of the forceps hole (the instrument channel) 18 and a focus position of the objective lens 17. In other words, the first gas feed outlet 33 is oriented toward the intersection O of the central axis of the instrument channel 18 and the focus position of the objective lens 17. In addition, the second gas feed hole 34 is arranged such that the gas is fed toward the objective lens 17.

Furthermore, as shown in FIG. 5, the tip hood 30 includes a marking 35a indicating the position of the forceps hole 18 and a marking 35b indicating the position of the objective lens 17. The markings 35a and 35b configure a forceps hole indicator (hereinafter also referred to as an instrument channel indicator) and an objective lens indicator, respectively. When attaching the tip hood 30 to the distal end portion 11a, the user aligns the marking 35a with the forceps hole 18 and aligns the marking 35b with the objective lens 17.

Such alignments cause the first gas feed hole 33 to be arranged to face the intersection O of the axial direction (more specifically, the central axis) of the forceps hole (the instrument channel) 18 and the focus position of the objective lens 17, and cause the second gas feed hole 34 to be arranged to face the objective lens 17. As a result, in the smoke removal, the tip hood 30 can be fixed to the distal end portion 11a of the endoscope 10 at the most efficient mounting angle.

### Modified Example

FIG. 6 shows a configuration of a tip hood according to a modified example of the first embodiment.

In the above-described embodiment, the tip hood 30 includes the first gas feed hole 33 and the second gas feed hole 34, but is not limited to such a configuration. For example, the first gas feed hole 33 and the second gas feed hole 34 may be provided at a plurality of locations.

As shown in FIG. 6, a tip hood 30A includes a plurality of first gas feed holes 33 and a plurality of second gas feed holes 34. A set of the first gas feed hole 33 and the second gas feed hole 34 is provided inside the tip hood 30A at three locations (for example, at every 120 degrees). Note that the first gas feed hole 33 and the second gas feed hole 34 may be provided at two locations, or four locations or more.

The three first gas feed holes 33 and the three second gas feed holes 34 are provided. However, the number of the first gas feed holes 33 and the number of the second gas feed holes 34 may be different from each other. For example, the first gas feed holes 33 may be provided respectively at three locations, and the second gas feed hole 34 may be provided at only one location.

The first gas feed holes 33 and the second gas feed holes 34, which are provided respectively at a plurality of locations, receive feeding of the gas branched from the gas feed conduit 31. Note that a plurality of gas feed conduits may be provided and the gas may be fed from the plurality of gas feed conduits to the first gas feed holes 33 and the second gas feed holes 34.

The tip hood 30A is thus provided with the plurality of first gas feed holes 33 and the plurality of second gas feed holes 34, which eliminates the need for considering the attaching position (angle) of the tip hood 30A with respect to the distal end portion 11a. In other words, even if the tip hood 30A is attached to the distal end portion 11a in any positional relationship, any of the plurality of first gas feed holes 33 is arranged so as to face the intersection O of the axial direction (more specifically, the central axis) of the forceps hole (the instrument channel) 18 and the focus position of the objective lens 17 and any of the plurality of second gas feed holes 34 is arranged so as to face the objective lens 17.

### Second Embodiment

Next, a second embodiment will be described.

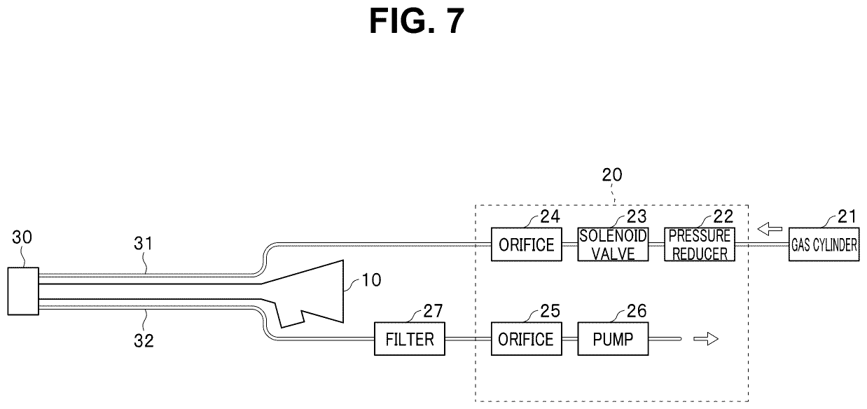

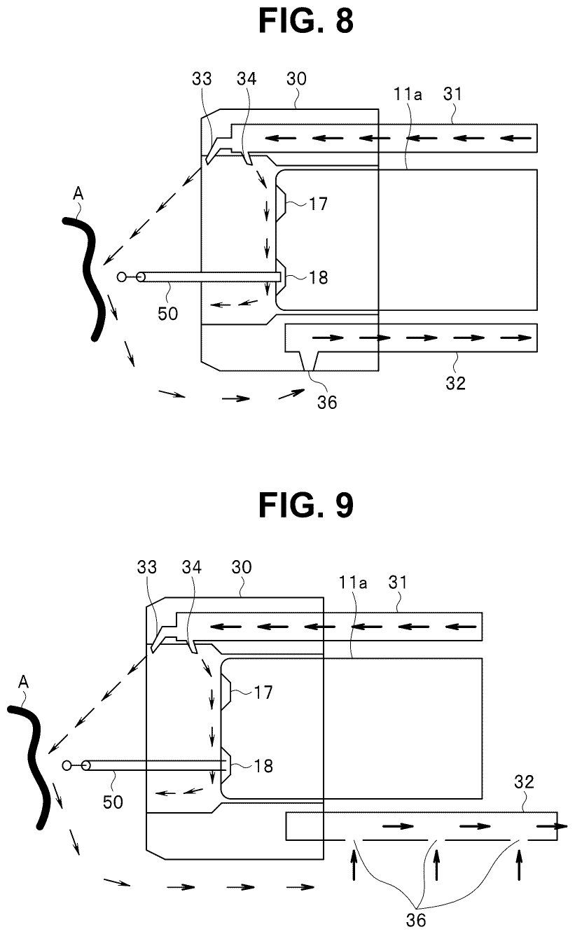

FIG. 7 shows a configuration of a smoke removal device in the second embodiment. In addition, FIG. 8 shows a configuration of a tip hood in the second embodiment.

A smoke removal device 20 may include processing circuitry including one or more processors and one or more non-transitory computer-readable storage media. For instance, the smoke removal device 20 includes: a pressure reducer 22 that reduces a pressure of a gas (CO₂ gas) supplied from a gas cylinder 21 which is a gas supply source to a pressure safe for a human body; a solenoid valve 23 that controls start and stop of gas feeding; an orifice 24 that adjusts a gas feed flow rate; an orifice 25 that adjusts a gas discharge flow rate; and a pump 26 that generates a gas discharge pressure. The pressure reducer 22, the solenoid valve 23, and the orifice 24 constitute a gas feeding portion, and the orifice 25 and the pump 26 constitute a gas discharging portion.

A gas feed conduit 31 is connected to the orifice 24 and a gas discharge conduit (hereinafter also referred to as a gas exhaust conduit) 32 is connected to the orifice 25. In addition, a filter 27 is arranged on the gas exhaust conduit 32. The filter 27 is configured to filter the smoke contained in the discharged gas, and inhibit toxic substances contained in the smoke from being released into the atmosphere.

The orifice opening sizes of the orifice 24 and the orifice 25 are adjusted such that the gas feed flow rate and the gas discharge flow rate are equal to each other. This inhibits fluctuation of an inner pressure in the digestive tract.

In general, the greater the gas feed flow rate, the higher the effect of the smoke removal. However, in a normal procedure using an endoscope, adjusting the orifice 24 such that the gas feed flow rate from the first gas feed hole 33 is approximately 0.5 to 3 L/min, and the gas feed flow rate from the second gas feed hole 34 is approximately 0.5 L/min provides a necessary and sufficient smoke removal effect.

In addition, from the perspective of safety, the gas feeding pressure (delivery pressure of the pressure reducer) may be suppressed to 40 kPa or below. Considering the insertability into the digestive tract, the gas feed conduit 31 is formed such that the inner diameter thereof is 2 mm or smaller and the outer diameter thereof is 3 mm or smaller. In this case, if the opening diameter of the first gas feed hole 33 is approximately 0.5 to 1 mm and the opening diameter of the second gas feed hole 34 is approximately 0.5 mm, the above-described flow rates can be ensured.

As shown in FIG. 8, the tip hood 30 includes a gas discharge hole (hereinafter also referred to as a gas exhaust port) 36. The gas exhaust port 36 is provided at an outer side of the tip hood 30. Note that the position where the gas exhaust port 36 is provided is not limited to the position shown in FIG. 8. The gas exhaust port 36 may be arranged at any position as long as it is provided outside the viewing angle of the endoscope 10, for example. The gas exhaust port 36 is provided outside the viewing angle of the endoscope 10, thereby inhibiting the smoke from being reflected when the gas is discharged.

In addition, the gas exhaust port 36 is provided at the tip hood 30, but is not limited to such a configuration. The gas exhaust port 36 may be provided in the gas exhaust conduit 32, for example. FIG. 9 shows a configuration in which the gas exhaust ports 36 are provided in the gas exhaust conduit 32.

As shown in FIG. 9, the gas exhaust ports 36 are provided respectively at three locations of the gas exhaust conduit 32. The number of the locations where the gas exhaust ports 36 are provided in the gas exhaust conduit 32 is not limited to three. The number of the locations may be one, two, or four or more. The gas exhaust ports 36 are thus provided dispersedly at a plurality of locations, to thereby enable the smoke scattered in the digestive tract to be discharged efficiently.

### Third Embodiment

Next, a third embodiment will be described.

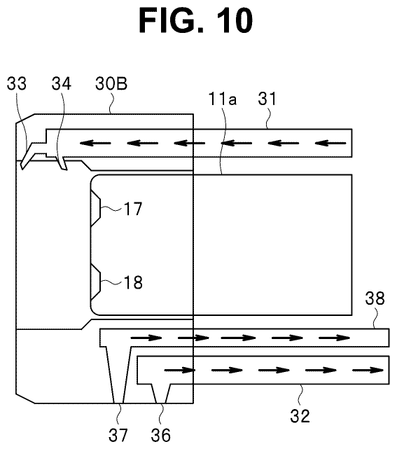

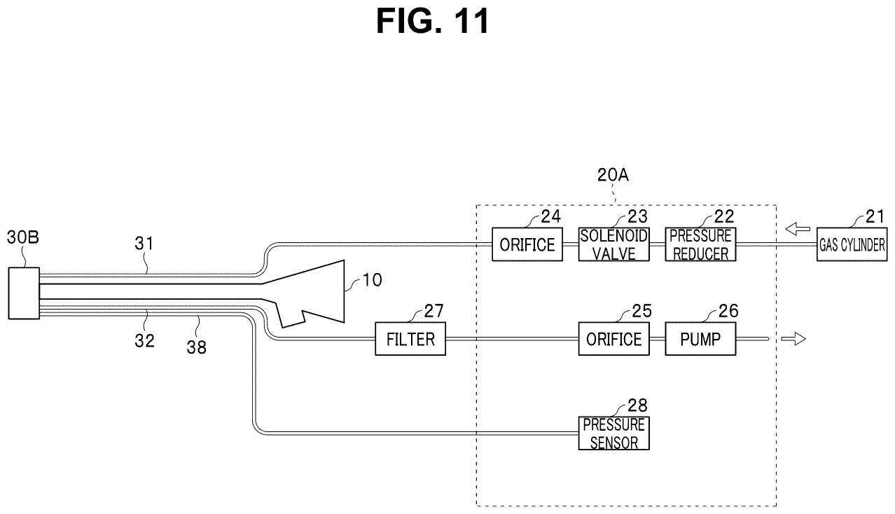

FIG. 10 shows a configuration of a tip hood in the third embodiment. In addition, FIG. 11 shows a configuration of a smoke removal device in the third embodiment.

As shown in FIG. 10, a tip hood 30B includes a pressure intake hole 37, and a pressure transmission conduit 38. A smoke removal device 20A shown in FIG. 11 may include processing circuitry including one or more processors and one or more non-transitory computer-readable storage media. For instance, the smoke removal device 20A includes a pressure sensor 28, in addition to a pressure reducer 22, a solenoid valve 23, an orifice 24, an orifice 25, and a pump 26.

The pressure intake hole 37 is provided at an outer side of the tip hood 30B. The pressure inside the tip hood 30B becomes higher than the pressure around it, due to gas feeding from the second gas feed hole 34. In view of this, the pressure intake hole 37 is provided at a position other than the inside of the tip hood 30B, for example, at the outer side of the tip hood 30B, as shown in FIG. 10, thereby enabling accurate measurement of the pressure in the digestive tract.

Note that the pressure intake hole 37 is provided at the tip hood 30B, but not limited to this configuration. The pressure intake hole 37 may be provided at the pressure transmission conduit 38, for example.

The pressure transmission conduit 38 is connected to the pressure sensor 28 of the smoke removal device 20A. The pressure sensor 28 is configured to measure the pressure inside the digestive tract through the pressure intake hole 37 and the pressure transmission conduit 38. When the pressure inside the digestive tract measured by the pressure sensor 28 exceeds a predetermined value, the smoke removal device 20A drives the pump 26, to discharge the gas from the inside of the digestive tract and decrease the pressure inside the digestive tract.

Note that the pressure sensor 28 is provided in the smoke removal device 20A, but is not limited to the configuration. For example, the pressure sensor 28 is provided in the tip hood 30B and the pressure sensor 28 and the smoke removal device 20A are connected by an electric cable. Then, information on the pressure measured by the pressure sensor 28 may be transmitted to the smoke removal device 20A through the electric cable.

Thus, the pressure inside the digestive tract is measured, and the pump 26 is driven depending on the measurement result, to thereby enable the pressure inside the digestive tract to be maintained at an appropriate value.

### Modified Example

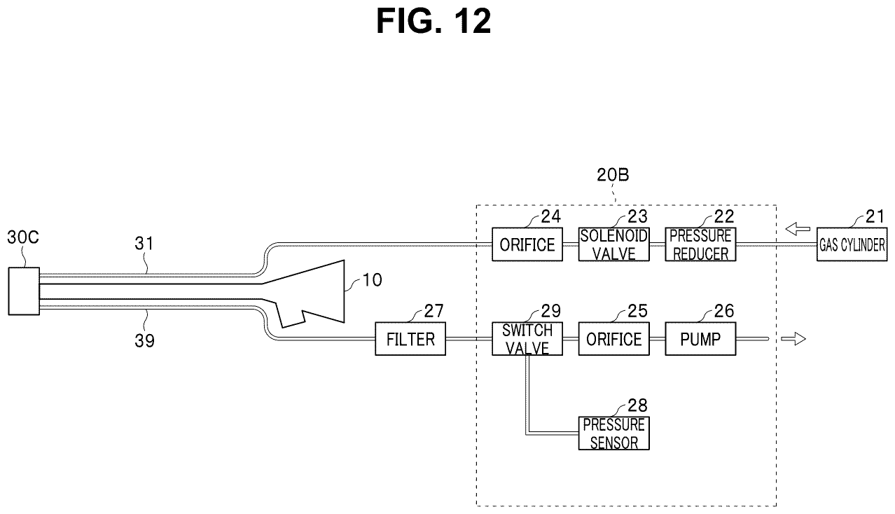

FIG. 12 shows configurations of a tip hood and a smoke removal device in a modified example of the third embodiment.

As shown in FIG. 12, a tip hood 30C includes a gas discharge/pressure transmission conduit 39. The gas discharge/pressure transmission conduit 39 is configured by forming the gas exhaust conduit 32 and the pressure transmission conduit 38 that are shown in FIG. 11 as a single common conduit.

A smoke removal device 20B shown in FIG. 12 may include processing circuitry including one or more processors and one or more non-transitory computer-readable storage media. For instance, the smoke removal device 20B is configured by adding a switch valve 29 to the smoke removal device 20A in FIG. 11. The gas discharge/pressure transmission conduit 39 is connected to the switch valve 29 provided in the smoke removal device 20B.

The switch valve 29 switches, based on the control by the smoke removal device 20B, the connection destination of the gas discharge/pressure transmission conduit 39 between the pressure sensor 28, and the orifice 25 and the pump 26.

The smoke removal device 20B connects the gas discharge/pressure transmission conduit 39 to the pressure sensor 28, and measures the pressure inside the digestive tract. Then, when the pressure measured by the pressure sensor 28 exceeds a predetermined value, the smoke removal device 20B connects the gas discharge/pressure transmission conduit 39 to the orifice 25 and the pump 26. This causes the gas to be discharged from the digestive tract, to lower the pressure inside the digestive tract.

Note that control may be performed to adjust the gas discharge amount such that the pressure inside the digestive tract is constant, by alternately performing the pressure measurement by the pressure sensor 28 and the gas discharge by the pump 26. The adjustment of the gas discharge amount is performed by changing the rotation speed of the pump 26, or using a predetermined valve, the opening degree of which can be adjusted, instead of the orifice 25.

Forming the gas exhaust conduit 32 and the pressure transmission conduit 38 that are shown in FIG. 10 and FIG. 11 as the single common conduit enables the size reduction of the tip hood 30C, which improves the insertability and the operability of the endoscope 10. In addition, when a body fluid or the like of a patient enters the pressure transmission conduit 38 shown in FIG. 10 and FIG. 11, it is difficult to remove the body fluid or the like. In contrast, when the body fluid or the like of the patient enters the gas discharge/pressure transmission conduit 39, the entered body fluid can be sucked and removed by switching the connection destination of the conduit to the orifice 25 and the pump 26.

### Fourth Embodiment

Next, a fourth embodiment will be described.

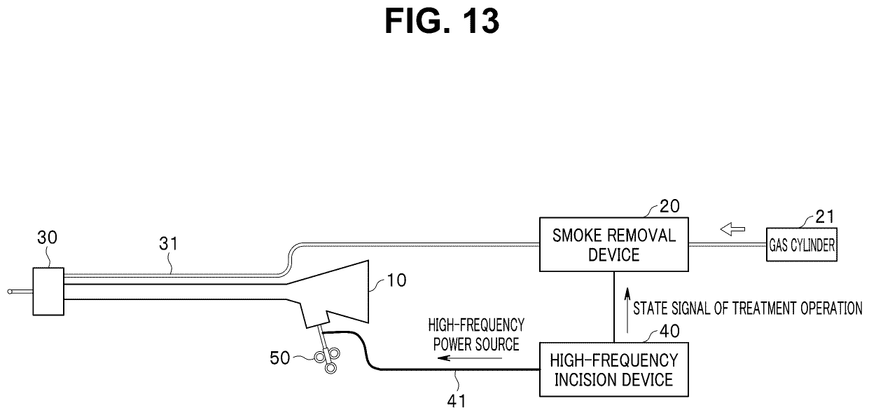

FIG. 13 shows a connection relationship between a smoke removal device and a high-frequency incision device in the fourth embodiment.

A smoke removal device 20 and a high-frequency incision device 40 are electrically connected to each other. The high-frequency incision device 40 transmits a state signal according to whether a treatment instrument 50 (e.g., a high-frequency probe) is in an output state to the smoke removal device 20 electrically connected to the high-frequency incision device 40.

The smoke removal device 20 may include processing circuitry including one or more processors and one or more non-transitory computer-readable storage media. For instance, the smoke removal device 20 is configured to perform, according to the state signal from the high-frequency incision device 40, gas feeding for smoke removal, only when the treatment instrument 50 is in the output state (i.e., an active state where it is supplied with high-frequency power and currently in use for treatment).

Normally, smoke is generated when a high-frequency signal is outputted to the treatment instrument 50 and treatment is performed on a diseased part A. Therefore, according to the state signal from the high-frequency incision device 40, the gas feeding for smoke removal is performed only when the treatment instrument 50 is in the output state.

The gas feeding is thus performed only in the state where smoke is being generated (or smoke is possibly generated), which enables suppression of a pressure increase in the digestive tract. In addition, such a control enables CO₂ gas consumption to be reduced, which leads to a reduction in the procedure costs.

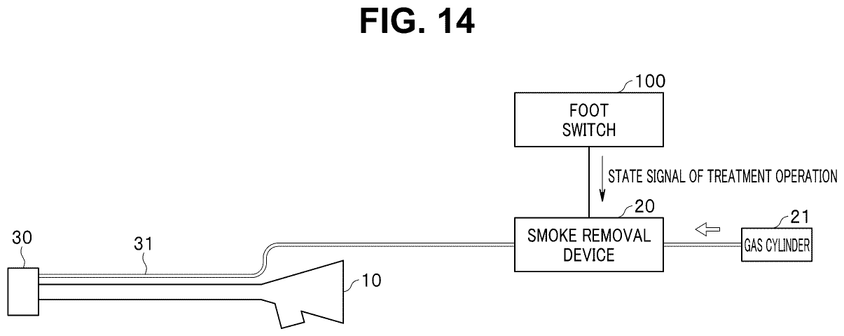

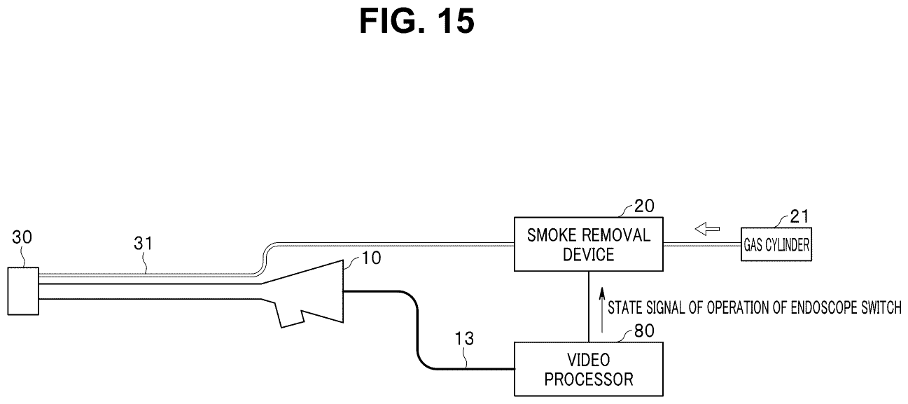

Note that, as shown in FIG. 14 and FIG. 15, start and stop of gas feeding (smoke removal) may be controlled by various switches.

FIG. 14 shows a configuration in a case where the start and stop of the smoke removal is controlled by a foot switch.

A foot switch 100 is connected to the smoke removal device 20. When a switch operation is performed on the foot switch 100, the foot switch 100 transmits a state signal of the switch operation to the smoke removal device 20.

The smoke removal device 20 stops the gas feeding for smoke removal when the foot switch 100 is not depressed, and starts the gas feeding for smoke removal when the foot switch 100 is depressed. Thus, the start and stop of the gas feeding for smoke removal is controlled by the foot switch 100 connected to the smoke removal device 20.

The gas feeding for smoke removal can be performed only in a situation where smoke is generated, based on a judgment of a user, which enables the pressure increase in the digestive tract to be suppressed. Furthermore, with such control, the CO₂ gas consumption can be reduced, which leads to a reduction in the procedure costs.

Note that a hand switch may be connected to the smoke removal device 20 instead of the foot switch 100, and according to a state signal of a switch operation performed on the hand switch, the start and stop of the gas feeding for smoke removal may be controlled.

Alternatively, according to a state signal of a switch operation performed on a switch provided at the endoscope 10, the start and stop of the gas feeding for smoke removal may be controlled.

FIG. 15 shows a configuration in a case where the start and stop of the smoke removal is controlled by the switch of the endoscope.

As shown in FIG. 15, the video processor 80 is electrically connected to the smoke removal device 20. When the switch of the endoscope 10 is operated, the state signal of the switch operation is transmitted to the smoke removal device 20 via the video processor 80.

The smoke removal device 20 stops the gas feeding for smoke removal when the switch of the endoscope 10 is not depressed, and starts the gas feeding for smoke removal when the switch of the endoscope 10 is depressed.

As a result, the gas feeding for smoke removal can be performed only in the situation where smoke is generated, based on the judgment of the user, which enables the pressure increase in the digestive tract to be suppressed. Furthermore, with such control, the CO₂ gas consumption can be reduced, which leads to a reduction in the procedure costs.

Note that the control of the start and stop of the gas feeding for smoke removal is not limited to be performed by operating the various switches.

An endoscopic image picked up by the endoscope 10 is inputted to the video processor 80. The video processor 80 performs image analysis on the endoscopic image inputted from the endoscope 10, to determine presence or absence of smoke. The video processor 80 then transmits a determination signal indicating the presence or absence of smoke to the smoke removal device 20.

The smoke removal device 20 controls the start and stop of the smoke removal based on the determination signal indicating the presence or absence of smoke which has been transmitted from the video processor 80. Specifically, the smoke removal device 20 starts the smoke removal when the video processor 80 determines the presence of the smoke, and stops the smoke removal when the video processor 80 determines that no smoke is present.

As a result, the gas feeding for smoke removal can be performed only in the case where it has been determined that smoke is generated, based on the endoscopic image, and thereby the pressure increase in the digestive tract can be suppressed. Furthermore, with such control, the CO₂ gas consumption can be reduced, which leads to a reduction in the procedure costs.

### Fifth Embodiment

Next, a fifth embodiment will be described.

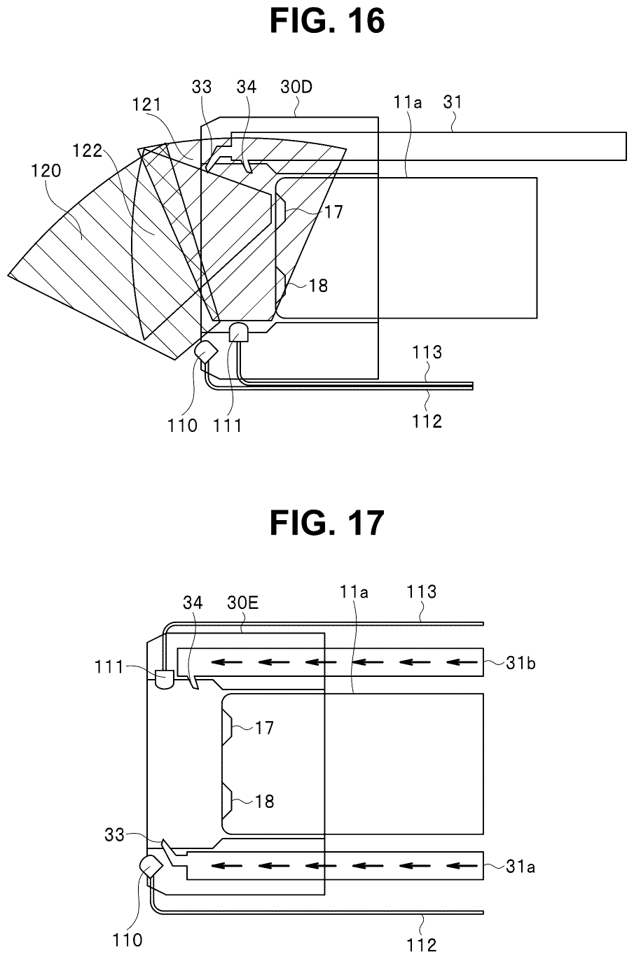

FIG. 16 shows a configuration of a tip hood in the fifth embodiment.

A tip hood 30D includes a first smoke sensor 110 and a second smoke sensor 111 that can detect smoke. The first smoke sensor 110 and the second smoke sensor 111 are connected to a smoke removal device 20 by electric wiring 112 and 113, respectively. Note that the first smoke sensor 110 and the second smoke sensor 111 may be other sensors, for example, heat sensors or optical sensors, as long as they can detect smoke.

The first smoke sensor 110 is arranged so as to detect the smoke in the field-of-view direction of the endoscope 10. In addition, the second smoke sensor 111 is arranged so as to detect the smoke on the distal end side (in the tip hood 30D) of the endoscope 10.

Specifically, the first smoke sensor 110 and the second smoke sensor 111 are arranged so as to cover a field-of-view range 122 of the endoscope 10 with a detection area 120 of the first smoke sensor 110 and a detection area 121 of the second smoke sensor 111. Note that the number of the smoke sensors arranged in the tip hood 30D is not limited to two. The number may be one or three or more as long as the field-of-view range 122 of the endoscope 10 can be covered.

The smoke removal device 20 starts gas feeding for smoke removal when smoke is detected by the first smoke sensor 110 and/or the second smoke sensor 111, and stops the gas feeding for smoke removal when no smoke is detected.

As a result, the gas feeding for smoke removal can be performed only in the situation where smoke is generated, which enables a pressure increase in the digestive tract to be suppressed. Furthermore, with such control, the CO₂ gas consumption can be reduced, which leads to a reduction in the procedure costs.

### Sixth Embodiment

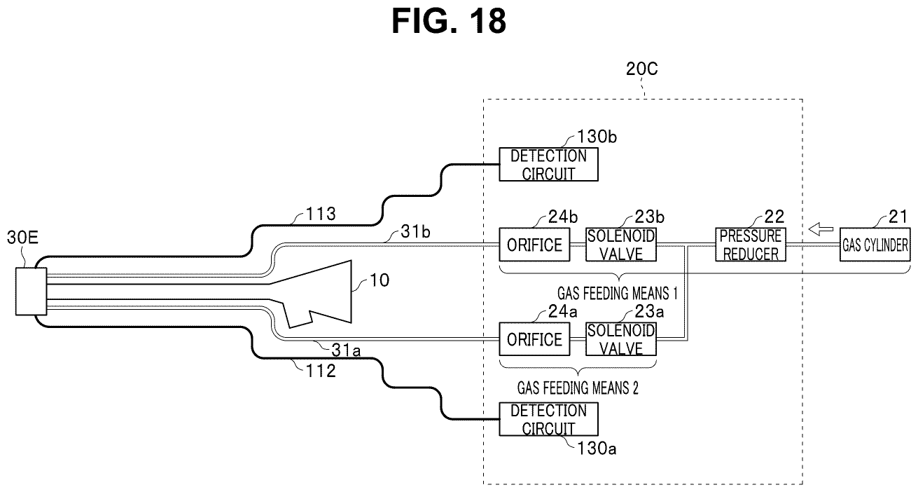

FIG. 17 shows a configuration of a tip hood in a sixth embodiment. In addition, FIG. 18 shows a configuration of a smoke removal device in the sixth embodiment.

A tip hood 30E and a smoke removal device 20C in the sixth embodiment are configured to control gas feeding from a first gas feed hole 33 and gas feeding from a second gas feed hole 34, independently. To this end, a first gas feed conduit 31a is connected to the first gas feed hole 33 and a second gas feed conduit 31b is connected to the second gas feed hole 34.

The smoke removal device 20C may include processing circuitry including one or more processors and one or more non-transitory computer-readable storage media. For instance, the smoke removal device 20C includes a pressure reducer 22, a first solenoid valve 23a, a second solenoid valve 23b, a first orifice 24a, a second orifice 24b, a first detection circuit 130a, and a second detection circuit 130b.

The first gas feed conduit 31a is connected to the first solenoid valve 23a through the first orifice 24a. In addition, the second gas feed conduit 31b is connected to the second solenoid valve 23b through the second orifice 24b.

The first smoke sensor 110 is connected to the first detection circuit 130a of the smoke removal device 20C through the electric wiring 112. In addition, the second smoke sensor 111 is connected to the second detection circuit 130b of the smoke removal device 20C through the electric wiring 113. The first detection circuit 130a detects the state of the first smoke sensor 110. The second detection circuit 130b detects the state of the second smoke sensor 111.

The smoke removal device 20C controls the first solenoid valve 23a when the first smoke sensor 110 detects smoke, and performs gas feeding from the first gas feed hole 33 via the first gas feed conduit 31a. On the other hand, the smoke removal device 20C controls the second solenoid valve 23b when the second smoke sensor 111 detects smoke, and performs gas feeding from the second gas feed hole 34 via the second gas feed conduit 31b. Furthermore, the smoke removal device 20C controls the first solenoid valve 23a and the second solenoid valve 23b when the first smoke sensor 110 and the second smoke sensor 111 detect smoke, and performs the gas feeding from the first gas feed hole 33 and the second gas feed hole 34.

The gas feeding from the first gas feed hole 33 and the gas feeding from the second gas feed hole 34 are thus controlled independently, thereby enabling a gas feed amount to be reduced, compared to the case where the gas feeding is performed simultaneously from the first gas feed hole 33 and the second gas feed hole 34.

As a result, the gas feeding for smoke removal can be performed individually according to the smoke generation site, which enables a pressure increase in the digestive tract to be suppressed. Furthermore, with such control, the CO₂ gas consumption can be reduced, which leads to a reduction in the procedure costs.

### Seventh Embodiment

Next, a seventh embodiment will be described.

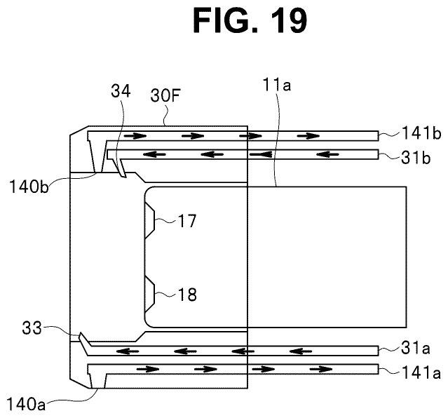

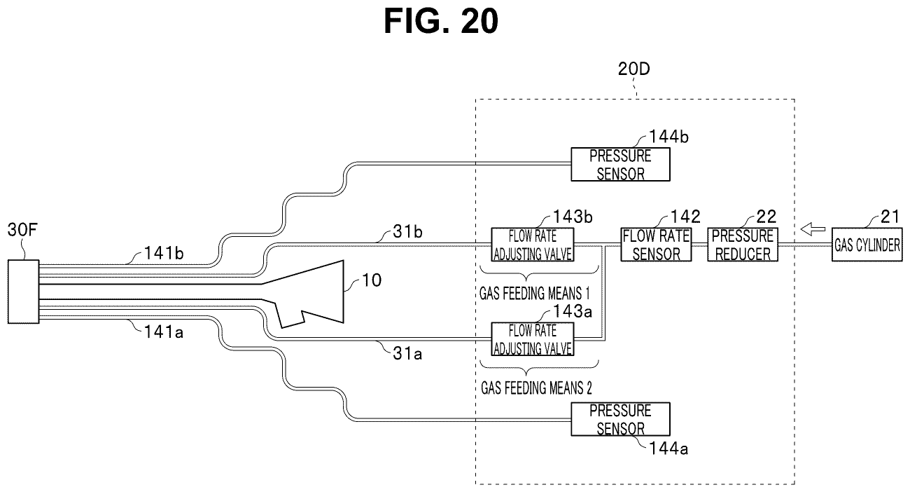

FIG. 19 shows a configuration of a tip hood in the seventh embodiment. In addition, FIG. 20 shows a configuration of a smoke removal device in the seventh embodiment.

A tip hood 30F and a smoke removal device 20D in the seventh embodiment are configured to control a gas feed amount (i.e., a flow rate of the gas to be fed) from a first gas feed hole 33 and/or a second gas feed hole 34 according to a difference between a pressure outside the tip hood 30F and a pressure inside the tip hood 30F.

The tip hood 30F includes a first pressure intake hole 140a, a first pressure transmission conduit 141a connected to the first pressure intake hole 140a, a second pressure intake hole 140b, and a second pressure transmission conduit 141b connected to the second pressure intake hole 140b.

The smoke removal device 20D may include processing circuitry including one or more processors and one or more non-transitory computer-readable storage media. For instance, the smoke removal device 20D includes a pressure reducer 22, a flow rate sensor 142, a first flow rate adjusting valve 143a, a second flow rate adjusting valve 143b, a first pressure sensor 144a, and a second pressure sensor 144b.

The first pressure intake hole 140a opens toward the outside of the tip hood 30F and is configured such that the pressure outside the tip hood 30F is taken in therethrough. On the other hand, the second pressure intake hole 140b opens toward the inside of the tip hood 30F and is configured such that the pressure inside the tip hood 30F is taken in therethrough.

The first pressure transmission conduit 141a and the second pressure transmission conduit 141b are connected respectively to the first pressure sensor 144a and the second pressure sensor 144b in the smoke removal device 20D. The first pressure sensor 144a measures the pressure outside the tip hood 30F. On the other hand, the second pressure sensor 144b detects the pressure inside the tip hood 30F.

The smoke removal device 20D controls a flow rate of a gas that is fed to the first gas feed conduit 31a and a flow rate of a gas that is fed to the second gas feed conduit 31b according to a difference between the pressure detected by the first pressure sensor 144a and the pressure detected by the second pressure sensor 144b.

Specifically, the first flow rate adjusting valve 143a and the second flow rate adjusting valve 143b, which can electrically adjust an orifice opening degree, are connected to the first gas feed conduit 31a and the second gas feed conduit 31b. The smoke removal device 20D controls the first flow rate adjusting valve 143a and the second flow rate adjusting valve 143b, to thereby control the flow rate of the gas that is fed to the first gas feed conduit 31a and the flow rate of the gas that is fed to the second gas feed conduit 31b.

In order to inhibit smoke from entering the tip hood 30F, the pressure inside the tip hood 30F needs to be maintained to be higher than the pressure outside the tip hood 30F. Specifically, the pressure inside the tip hood 30F may be approximately 0.5 mmHg higher than the pressure outside the tip hood 30F.

Therefore, the smoke removal device 20D controls, based on the detection results by the first pressure sensor 144a and the second pressure sensor 144b, the first flow rate adjusting valve 143a and the second flow rate adjusting valve 143b such that the pressure inside the tip hood 30F is equal to a value obtained by adding 0.5 mmHg to the pressure outside the tip hood 30F.

When the pressure inside the tip hood 30F is higher than the value obtained by adding 0.5 mmHg to the pressure outside the tip hood 30F, the smoke removal device 20D performs control so as to reduce the opening degree of the first flow rate adjusting valve 143a and increase the opening degree of the second flow rate adjusting valve 143b.

On the other hand, when the pressure inside the tip hood 30F is lower than the value obtained by adding 0.5 mmHg to the pressure outside the tip hood 30F, the smoke removal device 20D performs control so as to increase the opening degree of the first flow rate adjusting valve 143a and reduce the opening degree of the second flow rate adjusting valve 143b.

However, when the gas feed flow rate which is fed into the digestive tract is high, the pressure inside the digestive tract sharply increases. In view of this, the gas feed flow rates by the first flow rate adjusting valve 143a and the second flow rate adjusting valve 143b are measured by the flow rate sensor 142. Specifically, a total sum of the gas feed flow rate by the first flow rate adjusting valve 143a and the gas feed flow rate by the second flow rate adjusting valve 143b may be 0.5 to 3 L/min or less.

The smoke removal device 20D controls, based on the measurement result by the flow rate sensor 142, the first flow rate adjusting valve 143a and the second flow rate adjusting valve 143b such that the total sum of the gas feed flow rate by the first flow rate adjusting valve 143a and the gas feed flow rate by the second flow rate adjusting valve 143b is 0.5 to 3 L/min or less.

Note that the smoke removal device 20D may include two flow rate sensors such that the gas feed flow rate of the first flow rate adjusting valve 143a and the gas feed flow rate of the second flow rate adjusting valve 143b can be measured respectively by the two flow rate sensors. In addition, the smoke removal device 20D includes two pressure sensors, i.e., the first pressure sensor 144a and the second pressure sensor 144b, but is not limited to such a configuration. The smoke removal device 20D may be configured to measure a difference between the pressure outside the tip hood 30F and the pressure inside the tip hood 30F by using one differential pressure sensor.

As described above, the smoke removal device 20D is configured to control the gas feed amount from the first gas feed hole 33 and/or the second gas feed hole 34 according to the difference between the pressure outside the tip hood 30F and the pressure inside the tip hood 30F. With such a configuration, the pressure increase in the digestive tract can be suppressed while maintaining the effects of the smoke removal. Furthermore, with such control, the CO₂ gas consumption can be reduced, which leads to a reduction in the procedure costs.

### Eighth Embodiment

Next, an eighth embodiment will be described.

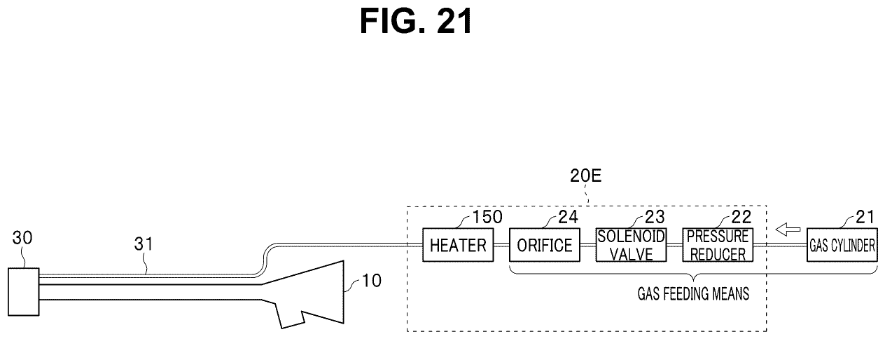

FIG. 21 shows a configuration of a smoke removal device in the eighth embodiment.

As described above, the inside of the digestive tract is in a high humidity environment, and if high-temperature smoke flows into the tip hood 30, moisture condensation occurs on the objective lens 17, which is likely to interfere with the field of view of the endoscope 10. In view of this, in the present embodiment, a smoke removal device 20E capable of inhibiting the moisture condensation on the objective lens 17 will be described.

The smoke removal device 20E may include processing circuitry including one or more processors and one or more non-transitory computer-readable storage media. For instance, the smoke removal device 20E includes a pressure reducer 22, a solenoid valve 23, an orifice 24, and a heater 150.

The heater 150 warms the CO₂ gas fed through the orifice 24 and feeds the warmed CO₂ gas to the gas feed conduit 31. This causes the warmed CO₂ gas to be sprayed from the second gas feed hole 34 toward the objective lens 17. As a result, the temperature of the objective lens 17 increases, which enables inhibition of the moisture condensation.

### Modified Example

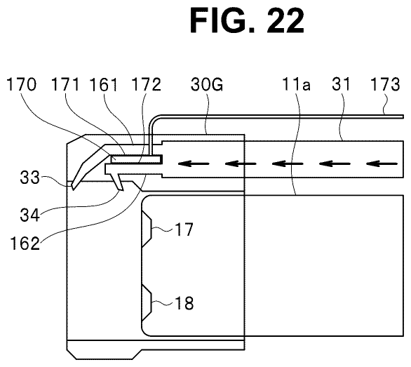

FIG. 22 shows a configuration of a tip hood in a modified example of the eighth embodiment.

A tip hood 30G includes a Peltier element 170 between a conduit 161 communicated with the first gas feed hole 33 and a conduit 162 communicated with the second gas feed hole 34.

The Peltier element 170 has a plate shape, and includes a first surface 171 that is in contact with the conduit 161 and a second surface 172 that is in contact with the conduit 162. In addition, electric wiring 173 for supplying a direct current is connected to the Peltier element 170.

The Peltier element 170 is a thermoelectric element, one surface of which absorbs heat (cools) and the other surface of which generates heat (heats) by the direct current being passed therethrough. In the present embodiment, the first surface 171 absorbs heat and the second surface 172 generates heat.

With such a configuration, the conduit 162 that is in contact with the second surface 172 is heated, to thereby be capable of warming the CO₂ gas which is sprayed from the second gas feed hole 34 toward the objective lens 17. As a result, the temperature of the objective lens 17 increases, which enables inhibition of the moisture condensation.

Furthermore, the conduit 161 that is in contact with the first surface 171 is cooled, to thereby be capable of cooling the CO₂ gas which is sprayed from the first gas feed hole 33 toward the smoke generation site. As a result, the temperature of the smoke is lowered, and thereby the moisture condensation on the objective lens 17 can be inhibited even if the smoke flows into the tip hood 30G.

Thus, the temperature of the generated smoke is lowered and the temperature of the objective lens 17 is increased by using the Peltier element 170, which increases the effect of inhibiting the moisture condensation on the objective lens 17 compared to the configuration in the eighth embodiment.

The present disclosure is not limited to the above-described embodiments, but various changes, combinations, and applications are possible within the scope without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), microcontrollers, digital signal processors (DSPs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, ROM, or other storage media) that stores data, software, firmware, or instructions contributing to the processing operations. Accordingly, the processing circuitry may be implemented as a specialized hardware circuit having fixed logic, a programmable circuit executing instructions stored in an internal or external memory, or any combination thereof. The processing circuitry may be configured as, or include, one or more processors. Thus, the term “processor” used in the description of embodiments is to be understood as a specific example of the processing circuitry or as a component included within the processing circuitry. Furthermore, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:
Clause 1: A tip hood comprising:
   a gas feed conduit for transmitting a predetermined gas;
   a first gas feed hole from which the gas is fed toward a field-of-view direction of an endoscope through the gas feed conduit; and
   a second gas feed hole from which the gas is fed toward a distal end side of the endoscope through the gas feed conduit.
Clause 2: The tip hood according to clause 1, wherein
   the first gas feed hole is bent from an axial direction of the gas feed conduit toward an inner surface.
Clause 3: The tip hood according to clause 1, wherein
   the second gas feed hole is bent from an axial direction of the gas feed conduit toward an inner surface.
Clause 4: An endoscope device comprising:
   an endoscope;
   a tip hood configured to be attached to a distal end portion of the endoscope;
   a gas feed conduit for transmitting a predetermined gas;
   a first gas feed hole which is provided at the tip hood and from which the gas is fed toward a field-of-view direction of the endoscope; and
   a second gas feed hole which is provided at the tip hood and from which the gas is fed toward a distal end side of the endoscope.
Clause 5: The endoscope device according to clause 4, further comprising:
   a forceps hole which is provided at a distal end of the endoscope and through which a treatment instrument is projected and retracted; and
   an objective lens provided at the distal end of the endoscope, wherein
   the first gas feed hole is arranged to orient in a direction of an intersection of an axial direction of the forceps hole and a focus position of the objective lens, and
   the second gas feed hole is arranged to orient in a direction of the objective lens.
Clause 6: The endoscope device according to clause 5, further comprising:
   a forceps hole indicator provided at the tip hood and configured to indicate a position of the forceps hole; and
   an objective lens indicator provided at the tip hood and configured to indicate a position of the objective lens.
Clause 7: The endoscope device according to clause 4, wherein
   the tip hood includes the first gas feed hole and the second gas feed hole, at a plurality of locations.
Clause 8: The endoscope device according to clause 4, wherein
   the tip hood includes a gas discharge hole through which a gas in a digestive tract is discharged, and
   a gas discharge conduit communicated with the gas discharge hole.
Clause 9: An endoscope system comprising:
   an endoscope;
   a tip hood configured to be attached to a distal end portion of the endoscope;
   a gas feed conduit for transmitting a predetermined gas;
   a control device configured to supply the predetermined gas to the gas feed conduit;
   a first gas feed hole which is provided at the tip hood and from which the gas is fed toward a field-of-view direction of the endoscope; and
   a second gas feed hole which is provided at the tip hood and from which the gas is fed toward a distal end side of the endoscope.
Clause 10: The endoscope system according to clause 9, further comprising
   a high-frequency incision device configured to control ON-OFF of an output of a treatment instrument, wherein
   the control device controls start and stop of transmission of the gas to the gas feed conduit in conjunction with information on an output state of the treatment instrument, the information being transmitted from the high-frequency incision device.
Clause 11: The endoscope system according to clause 10, wherein
   the control device performs gas feeding for smoke removal only when the treatment instrument is in the output state.
Clause 12: The endoscope system according to clause 10, wherein
   the control device performs gas feeding for smoke removal only in a state where an operation signal from an operation member that operates the treatment instrument is in an output state.
Clause 13: The endoscope system according to clause 9, further comprising an image processing apparatus configured to analyze an image picked up by the endoscope, wherein
   the control device controls start and stop of transmission of the gas to the gas feed conduit, based on an analysis result indicating presence or absence of smoke that is obtained by the image processing apparatus.
Clause 14: The endoscope system according to clause 9, wherein
   the control device controls gas feeding from the first gas feed hole and gas feeding from the second gas feed hole independently from each other.
Clause 15: The endoscope system according to clause 9, further comprising:
   a first smoke sensor provided at the tip hood and configured to detect a smoke generation site; and
   a second smoke sensor provided at the tip hood and configured to detect smoke inside the tip hood, wherein
   the control device controls gas feeding from the first gas feed hole based on a detection result by the first smoke sensor and independently controls gas feeding from the second gas feed hole based on a detection result by the second smoke sensor.
Clause 16: The endoscope system according to clause 9, further comprising:
   a first solenoid valve configured to control gas feeding from the first gas feed hole; and
   a second solenoid valve configured to control gas feeding from the second gas feed hole, wherein
   the control device controls the gas feeding from the first gas feed hole and the gas feeding from the second gas feed hole independently from each other by ON-OFF control of the first solenoid valve and the second solenoid valve.
Clause 17: The endoscope system according to clause 14, wherein
   the control device controls a gas feed amount from the first gas feed hole and/or the second gas feed hole according to a difference between a pressure outside the tip hood and a pressure inside the tip hood.
Clause 18: The endoscope system according to clause 14, wherein
   the tip hood includes a first pressure intake hole through which a pressure outside the tip hood is taken in and a second pressure intake hole through which a pressure inside the tip hood is taken in.
Clause 19: The endoscope system according to clause 9, wherein
   the control device includes a heater configured to warm the gas that is fed from the first gas feed hole and the second gas feed hole.
Clause 20: The endoscope system according to clause 9, wherein
   the tip hood includes a thermoelectric element configured to cool the gas that is fed from the first gas feed hole and warm the gas fed from the second gas feed hole.

What is claimed is:

1. A tip hood configured to be attached to a distal end portion of an endoscope, comprising:
a gas feed conduit configured to allow a predetermined gas to flow therethrough;
a first gas feed outlet configured to feed the gas, supplied through the gas feed conduit, in a field-of-view direction of the endoscope; and
a second gas feed outlet configured to feed the gas, supplied through the gas feed conduit, toward a distal end surface of the endoscope.

2. The tip hood according to claim 1, wherein
the first gas feed outlet is angled toward an interior of the tip hood with respect to an axial direction of the gas feed conduit.

3. The tip hood according to claim 1, wherein
the second gas feed outlet is angled toward an interior of the tip hood with respect to an axial direction of the gas feed conduit.

4. An endoscope device comprising:
an endoscope;
a tip hood configured to be attached to a distal end portion of the endoscope;
a gas feed conduit configured to allow a predetermined gas to flow therethrough;
a first gas feed outlet disposed at the tip hood and configured to feed the gas, supplied through the gas feed conduit, in a field-of-view direction of the endoscope; and
a second gas feed outlet disposed at the tip hood and configured to feed the gas, supplied through the gas feed conduit, toward a distal end surface of the endoscope.

5. The endoscope device according to claim 4, further comprising:
an instrument channel disposed at a distal end of the endoscope and configured to allow a treatment instrument to be advanced and retracted therethrough; and
an objective lens disposed at the distal end of the endoscope, wherein
the first gas feed outlet is oriented toward an intersection of a central axis of the instrument channel and a focus position of the objective lens, and
the second gas feed outlet is oriented toward the objective lens.

6. The endoscope device according to claim 5, further comprising:
an instrument channel indicator disposed at the tip hood to indicate a position of the instrument channel; and
an objective lens indicator disposed at the tip hood to indicate a position of the objective lens.

7. The endoscope device according to claim 4, wherein
the tip hood comprises:
the first gas feed outlet at a plurality of locations; and
the second gas feed outlet disposed at a plurality of locations.

8. The endoscope device according to claim 4, wherein
the tip hood comprises:
a gas exhaust port configured to allow a gas in a digestive tract to be exhausted therethrough; and
a gas exhaust conduit in communication with the gas exhaust port.

9. An endoscope system comprising:
an endoscope;
a tip hood configured to be attached to a distal end portion of the endoscope;
a gas feed conduit configured to allow a predetermined gas to flow therethrough;
a control device configured to control supply of the gas to the gas feed conduit;
a first gas feed outlet disposed at the tip hood and configured to feed the gas, supplied through the gas feed conduit, in a field-of-view direction of the endoscope; and
a second gas feed outlet disposed at the tip hood and configured to feed the gas, supplied through the gas feed conduit, toward a distal end surface of the endoscope.

10. The endoscope system according to claim 9, further comprising a high-frequency incision device configured to supply high-frequency power to a treatment instrument, wherein
the control device is further configured to:
receive, from the high-frequency incision device, information indicating whether the treatment instrument is in an active state where the high-frequency power is supplied thereto; and
control to start and stop supplying the gas to the gas feed conduit in conjunction with the received information.

11. The endoscope system according to claim 10, wherein
the control device is further configured to supply the gas for smoke removal only when the treatment instrument is in the active state.

12. The endoscope system according to claim 10, wherein
the control device is further configured to supply the gas for smoke removal only when an operation member configured to perform ON-OFF control of the treatment instrument is in an ON state where an ON signal is output from the operation member.

13. The endoscope system according to claim 9, further comprising
an image processing apparatus configured to analyze an image captured by the endoscope, wherein
the control device is further configured to:
receive, from the image processing apparatus, an analysis result indicating presence or absence of smoke; and
control to start and stop supplying the gas to the gas feed conduit based on the received analysis result.

14. The endoscope system according to claim 9, wherein
the control device is further configured to independently control gas feeding from the first gas feed outlet and gas feeding from the second gas feed outlet.

15. The endoscope system according to claim 9, further comprising:
a first smoke sensor disposed at the tip hood and configured to detect smoke at a smoke generation site; and
a second smoke sensor disposed at the tip hood and configured to detect smoke inside the tip hood, wherein
the control device is further configured to independently control:
gas feeding from the first gas feed outlet based on a detection result from the first smoke sensor; and
gas feeding from the second gas feed outlet based on a detection result from the second smoke sensor.

16. The endoscope system according to claim 9, further comprising:
a first solenoid valve configured to control gas feeding from the first gas feed outlet; and
a second solenoid valve configured to control gas feeding from the second gas feed outlet, wherein
the control device is further configured to independently control:
gas feeding from the first gas feed outlet by ON-OFF control of the first solenoid valve; and
gas feeding from the second gas feed outlet by ON-OFF control of the second solenoid valve.

17. The endoscope system according to claim 14, wherein
the control device is further configured to control at least one of a first flow rate of the gas to be fed from the first gas feed outlet or a second flow rate of the gas to be fed from the second gas feed outlet, based on a difference between a pressure outside the tip hood and a pressure inside the tip hood.

18. The endoscope system according to claim 14, wherein
the tip hood comprises:
a first pressure intake port configured to introduce a pressure outside the tip hood therethrough; and
a second pressure intake port configured to introduce a pressure inside the tip hood therethrough.

19. The endoscope system according to claim 9, wherein
the control device comprises a heater configured to warm the gas to be fed from the first gas feed outlet and the second gas feed outlet.

20. The endoscope system according to claim 9, wherein
the tip hood comprises a thermoelectric element configured to cool the gas to be fed from the first gas feed outlet and warm the gas to be fed from the second gas feed outlet.
